(19) United States
(12) Patent Application Publication
(10) Pub. No.: US 2023/0152222 A1
(43) Pub. Date: May 18, 2023

(54) DAIRY HERD IMPROVEMENT TESTING METHOD AND SYSTEM

(71) Applicants: MICHAEL MCHUGH, PLYMOUTH, MN (US); CHRISTOPHER ANDERSON, MINNEAPOLIS, MN (US)

(72) Inventors: MICHAEL MCHUGH, PLYMOUTH, MN (US); CHRISTOPHER ANDERSON, MINNEAPOLIS, MN (US)

(21) Appl. No.: 17/985,862

(22) Filed: Nov. 13, 2022

Related U.S. Application Data
(60) Provisional application No. 63/279,170, filed on Nov. 14, 2021.

(51) Int. Cl. G01N 21/359; G01N 21/3577; G01N 33/04; A01J 5/013

(52) U.S. Cl. CPC G01N 21/359; G01N 21/3577; G01N 33/04; A01J 5/0135

(57) ABSTRACT

Described herein is a system and a method to conduct for inline estimation of milk parameters such as fat, protein, lactose, somatic cell contents (SCC), and progesterone during the milking process that can be performed real time during the milking process with a commercially acceptable level of accuracy. In one example embodiment, specific wavelengths are identified that facilitate the use of low-cost Near-Infrared (NIR) Spectrometers and sensors to develop the inline, real time estimation system, with at least two segments or ranges being identified of NIR wavelengths for determining content or composition for these key parameters.

### CLAIM OF PRIORITY

This application claims priority to and the benefit of U.S. Provisional Application with Ser. No. 63/279170, filed on Nov. 14, 2021, with the same title, which is herein incorporated by reference in its entirety.

### FIELD OF THE INVENTION

The invention relates generally to analyzing a milk sample from a dairy cow.

### BACKGROUND

Poor reproductive performance is one of the most costly and difficult problems for dairy and livestock producers. Even in some well-managed herds, reproductive failure continues to be one of the primary reasons why cows are culled. Depending on the level of milk production in the herd and variable costs associated with poor reproductive management, a dairy producer loses between $1 to $3 per cow each day the cow is open (not pregnant) beyond the 90 days post-calving. Inaccurate or inefficient heat detection is still the major cause of low conception rates and long calving intervals. One tool or approach frequently used to help herd managers and veterinarians troubleshoot causes of poor reproductive performance—especially problems associated with heat detection—has been milk progesterone analysis. Scientists have tested milk samples for many years to monitor progesterone levels in cattle. In some cases, the assay they use is a very precise radioimmunoassay, requiring sophisticated equipment, radioactive tracers, and skilled technicians to perform the analysis, but cost and time delay create barriers for dairy farmers.

Currently record keeping or dairy activity monitors are used to indicate behaviors of readiness for breeding but do not inform users of the confirmation found in progesterone levels. Significant money is spent to time the breeding of a cow during a challenging window of progesterone. Additionally dairy cows are required to have monthly milk tests of five (5) commercially desired components (e.g., fat, protein, lactose, somatic cell contents (SCC), and progesterone) and the methods require labor, and time and shipping to send samples to a lab and wait for days. Therefore, there currently exists a need in the market for an analysis system and method that cost effectively speeds up the process of obtaining the above-mentioned key data and reduces the overall costs of required testing and data analysis.

### SUMMARY OF THE INVENTION

It would be advantageous to have a system and a method to conduct for inline estimation of milk parameters such as fat, protein, lactose, somatic cell contents (SCC), and progesterone during the milking process that can be performed real time during the milking process with a commercially acceptable level of accuracy. In one example embodiment, specific wavelengths are identified that facilitate the use of low-cost Near-Infrared (NIR) Spectrometers and sensors to develop the inline, real time estimation system. In general, two segments or ranges of NIR wavelengths were identified for determining content or composition for fat, protein, somatic cell content (SCC), and lactose. For progesterone, however, only wavelengths in the shorter segment or range were found to for a commercially viable inline estimation system. Progesterone levels may be a useful parameter in determining heat detection or estrus and early pregnancy in cattle, with high levels of progesterone indicating that the individual cow is not in estrus and low levels indicating that it is not pregnant.

A device attached in, on or near an inline milking unit to test and identify and report real time dairy milk components such as progesterone to determine these tests can help determine (1) if a cow is near estrus and potentially could conceive if bred, or (2) as an early indicator of pregnancy, as well as milk quality and general condition of the cow. Unlike the prior art, testing is performed during the milking process to measure for the above-mentioned components, using differing but specific NIR wavelengths proven to detect each of these components and measurements. In a related embodiment, the milk component measurement system also offers options to communicate measurements to a lab or a user wirelessly using radio frequency (RF). Further, unlike the prior art, the inventive concept discussed herein requires no manual handling or labor during use as it is automated and can operate on either AC or DC power.

Once attached inline as part of the equipment, NIR spectrometers can estimate milk composition in real-time during the entire period of milking, which usually lasts for an average of five minutes, as well as in the morning and evening. The shorter wavelengths of NIR are more suitable for the project, keeping in mind ease of measurements by using wider tube or light path lengths, and also because they can be sensed by inexpensive optical sensors. It will still bring unprecedented precision to a dairy farm and improve the quality of milk and cattle health.

In one example embodiment, there is provided a method of analyzing dairy cow milk components in a dairy cow milking system which include the steps of collecting a milk sample in line from a dairy cow using a transparent conduit and exposing the milk sample to a near infrared light source and at least one optical sensor module having a range of about 700 nm to about 1200 nm. The method also includes detecting substantially via transmittance and in real time a set of predetermined components within the milk sample, the predetermined components related to measurements or data generated from the at least one optical sensor module and the step of transmitting wirelessly the data from the optical sensor module to a microprocessor module. In an example embodiment, the microprocessor module is configured to generate the set of the set of predetermined components, where the set of predetermined milk components include one or more of protein, fat, vitamins, progesterone, and somatic cell count. These milk components indicate bovine conditions that affect milk production including one or more of mastitis, estrus, dehydration, and starvation.

In another example embodiment, there is provided a system for analyzing milk components in a dairy cow milking system that includes a transparent milk collection vessel or conduit and a suction apparatus having an inlet and an outlet, the outlet coupled to the milk collection vessel and the inlet adapted to be coupled to a dairy cow. Further included is a near infrared (NIR) spectrometer configured to provide light to and collect light, in a range of about 700 nm to about 1200 nm, from the milk collection vessel; and also included a controller module including a microcontroller and a memory module, the controller module adapted to receive data from the NIR spectrometer indicative of data measurements of a set of predetermined milk components. In a related embodiment, the system further includes a radio frequency (RF) wireless communications module configured to transmit data of at least one of the set of predetermined milk components including fat, protein, lactose, somatic cell contents (SCC), and progesterone.

The invention now will be described more fully hereinafter with reference to the accompanying drawings, which are intended to be read in conjunction with both this summary, the detailed description and any preferred and/or particular embodiments specifically discussed or otherwise disclosed. This invention may, however, be embodied in many different forms and should not be construed as limited to the embodiments set forth herein; rather, these embodiments are provided by way of illustration only and so that this disclosure will be thorough, complete and will fully convey the full scope of the invention to those skilled in the art.

### BRIEF DESCRIPTION OF THE DRAWINGS

FIG. 1 illustrates a schematic of a system of performing an inline real time analysis of a milk sample within a dairy cow milking system.

FIG. 2 illustrates a flow chart of a method of performing an inline real time analysis of a milk sample while using a dairy cow milking system.

FIG. 3 is a table that illustrates reproducibility (R) limits for milk analysis (laboratory, at-line, and in-line recommendations) and percentage of NIR prediction residuals (RES) equal to or below R (RES≤R, %) known in the prior art.

FIG. 4A is a graph that illustrates mean-centered first Savitzky-Golay derivative of absorbance derived from transmittance spectra (400-2,450 nm) of 300 raw milk samples, with the most important absorption bands for fat and CP, known in the prior art.

FIG. 4B is a graph that illustrates milk fat content determination discussed in the prior art.

FIG. 4C is a graph that illustrates milk total protein content determination discussed in the prior art.

FIG. 4D is a graph that illustrates lactose content determination discussed in the prior art.

FIG. 4E1 is a graph that illustrates a Near-infrared (NIR) spectra (851 to 1,649 nm) of raw milk from an in-line measurement setup in diffuse reflectance mode, where typical spectra of milk during 1 cow milking (n=12).

FIG. 4E2 is a graph illustrating an NIR spectra of all raw milk samples (n=785).

FIG. 4E3 is a graph illustrating a normalized NIR reflectance spectra of all raw milk samples (n=785).

### DETAILED DESCRIPTION OF THE INVENTION

Following are more detailed descriptions of various related concepts related to, and embodiments of, methods and apparatus according to the present disclosure. It should be appreciated that various aspects of the subject matter introduced above and discussed in greater detail below may be implemented in any of numerous ways, as the subject matter is not limited to any particular manner of implementation. Examples of specific implementations and applications are provided primarily for illustrative purposes.

The various embodiments of the invention provide for a system and a method to conduct for inline estimation of milk parameters such as fat, protein, lactose, somatic cell contents (SCC), and progesterone that can be performed real time, using an NIR spectroscopy-based system, during the milking process exhibiting commercially acceptable levels of accuracy. Generally, the NIR spectrum lies between 700 to 2500 nanometers (nm). This broad range can be divided into two segments of NIR wavelengths: the shorter wavelengths segment from 700-1100 nm, and the longer wavelengths between 1100-2500 nm. However, for practical and cost-effective implementations, it may not be necessary to include the entire range from 700-2500 nm in the milk composition estimation, since there appears to be no measurable improvement in accuracy when the two segments are combined. The NIR spectrum of longer wavelengths between 1000-2500 nm appears to give the best results, which are close to the standard laboratory method. Conversely, longer NIR wavelengths do appear to have two disadvantages: optical sensors needed for them are expensive; and the optimal path length of the light is 0.5 to 1 mm. The small width makes it unsuitable for inline measurements and a bypass is necessary. When used for online measurements, care must be taken that a collection or analysis tube or vessel used is narrow.

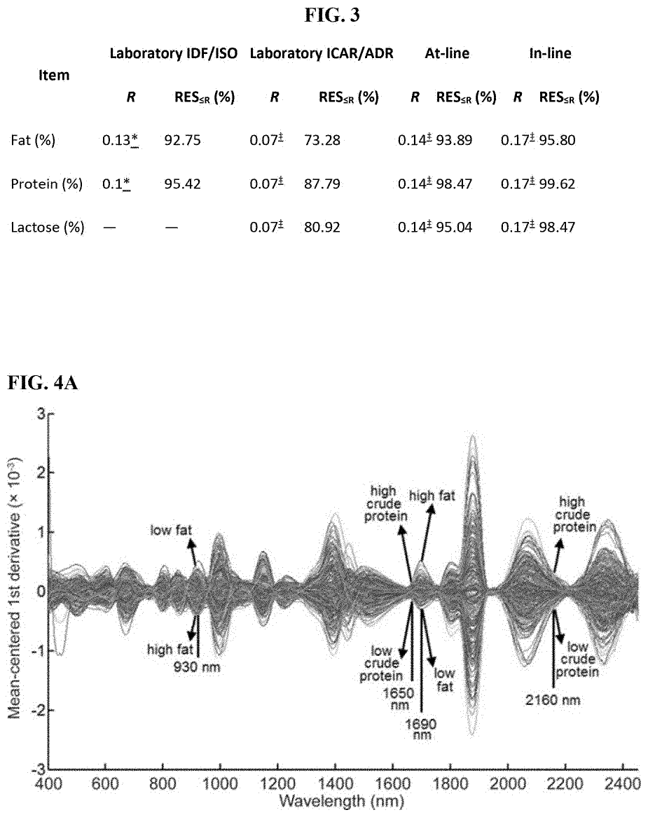

In contrast, shorter wavelengths, use a light pathway between 10 to 13.5 mm, so the online/inline bypass tube can be wider than when longer wavelengths are used. The optical sensors for the segment of shorter NIR wavelengths are inexpensive. The at-line measurements by shorter wavelengths (700 to 1100 nm) are comparable in accuracy to the 1100-2500 nm range. The online estimations by the shorter wavelengths are satisfactory since it appears that the lower accuracy by shorter wavelengths, during online and inline estimations, is commercially acceptable and is to be expected. Even various standards set by international and national agencies, for milk quality, like the ICAR or ISO accept lower accuracy for inline estimations in comparison to laboratory procedures. See FIG. 3 (Table 1), where the error (Res_{R}) for inline can be higher than laboratory or at-line results. In particular, FIG. 3 illustrates reproducibility (R) limits for milk analysis (laboratory, at-line, and in-line recommendations) and percentage of NIR prediction residuals (RES) equal to or below R (RES≤R, %). Symbols indicate the reference(s) corresponding to the value of R: an asterisk (*) indicates adapted according to IDF standard 141C:2000 (IDF, 2000) and ISO standard 9622. (ISO, 1999; SD of fat: 0.045%; SD of protein: 0.035%); a plus sign (+) indicates ADR recommendation 1:13 (ADR, 2002); a dagger (‡) indicates the ICAR standard (ICAR, 2010); a bullet (⋅) indicates adapted according to the ICAR (2010) and ADR (2002) recommendations,” Aernouts et al 2011. (Credits: DOI:https://doi.org/10.3168/jds.2011-4354)

Online measurements can monitor individual cows daily, which would give more information to help decision-making by dairy owners, than a perfect estimation done once in two to three weeks. The shorter NIR wavelengths are suitable for spectroscopy estimations as they will allow for rapid and non-destructive milk composition estimation in real-time and improve the quality and quantity of dairy milk production.

The mode of NIR sensor measurement commonly reported for milk is diffuse transmittance. Data is collected as transmittance spectra and recorded in the linked computer as absorbance [i.e., log (1/T)]. In some cases, reflectance can give better results for fat and protein measurements in the 1100-2500 nm range, however, lactose is more challenging for estimating by reflectance accurately. So, the transmittance mode is better than reflectance to find milk composition. The suitable wavelengths chosen are those that showed the most variations for each parameter. In other words, these wavelengths are sensitive to changes in concentrations of the respective parameter. For the NIR region from 700 to 1100 nm, where inexpensive online sensors could be used, the highest positive coefficients for measuring the various parameters are given.

Fat Estimation—Mode: Data is collected as transmittance spectra at 2-nm intervals and recorded in the linked computer as absorbance [i.e., log (1/T)]. Spectral data collection can have a path length up to 10 mm. Best Wavelengths: To estimate fat, the best wavelengths to use are 730, 770, 930, 968, 990, 1026, 1076, and 1092 nm; 930 nm was the best.

Lactose-Mode: Data is collected as transmittance spectra at 2-nm intervals and recorded in the linked computer as absorbance [i.e., log (1/T)]. Spectral data collection can have a path length up to 10 mm. Best Wavelength: For lactose, the best wavelengths are 734, 750, 786, 812, 908, 974, 982, and 1064 nm; and 1064 nm were the best.

Protein-Mode: Data is collected as transmittance spectra at 2-nm intervals and recorded in the linked computer as absorbance [i.e., log (1/T)]. Spectral data collection can have a path length up to 10 mm. Best Wavelength: To estimate proteins use 726,736, 760, 776, 880, 902, 952, and 1034 nm.

Progesterone—There is only one study, so far, to estimate progesterone in milk using NIR spectroscopy by Iweka et al 2020. This data is for inline estimation. Mode: Use absorbance data between 700-1050 nm at 1-nm intervals. Wavelengths: The two most important wavelengths are 740 and 840 nm. But these wavelengths are also said to be relevant for fat, proteins, and lactose. No further details are available for any other parameters by these scientists, except that they used the spectrum between 700-1100 nm.

SCC (somatic cell count)—Two studies successfully estimated SCC using the short-wavelength segment, but do not mention the specific NIR wavelengths they found useful. A third study was found that makes recommendations in the shorter wavelength range. The results obtained were within the range needed for daily real-time monitoring of milk for mastitis detection. Mode: Use transmittance data between 700-1100 nm at 2-nm intervals. The sample size used was 10 ml of milk in a test tube with 12 mm inner diameter and 16 mm outer diameter. So the light length that can be used is 16 mm. Wavelengths: The best positive correlations are 832, 926, and 960 nm. The highest negative correlation was at 1004 nm. Other important wavlengths are 762, 780, and 870 nm. The results reflect the changes in protein and fat that occurs due to changes in health of the cows.

Recommendations for Longer NIR Wavelengths Segment—Since the region of longer NIR wavelengths (1100 to 2400 nm), gives more precise results, these findings are also mentioned here, in case it is chosen for use. Results from two studies are covered below.

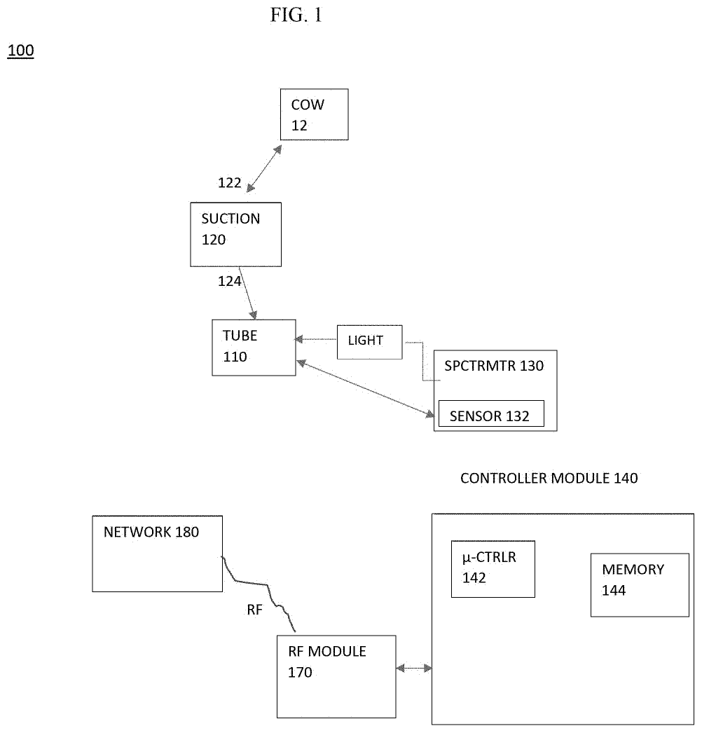

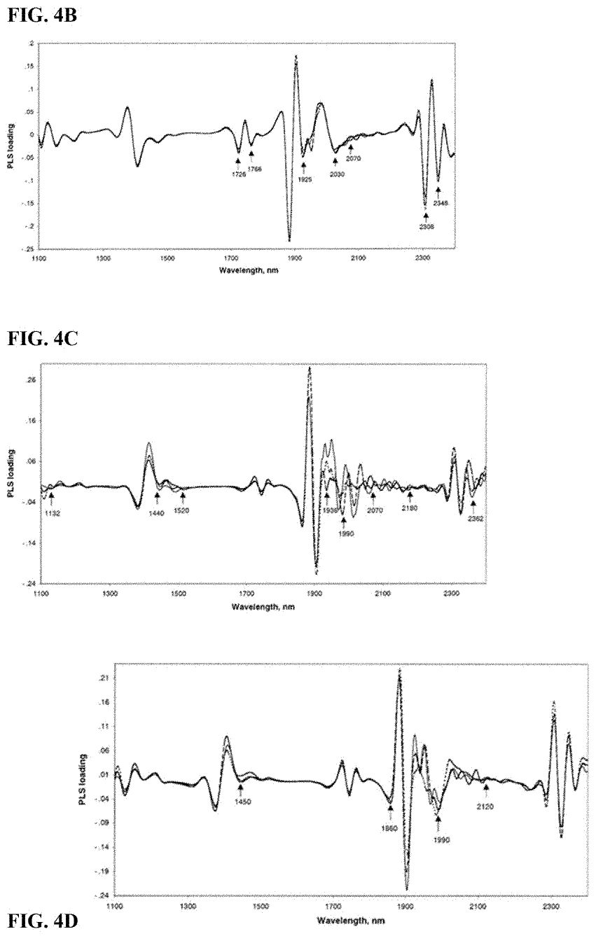

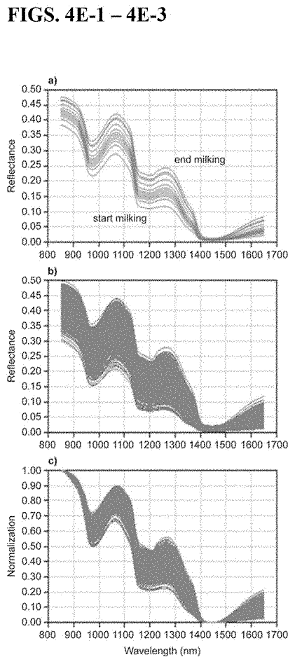

Fat Estimation—Mode: Data is collected as transmittance spectra at 2-nm intervals and recorded in the linked computer as absorbance [i.e., log (1/T)], in both studies (see FIG. 4A). FIG. 4A (FIG. 1 in the article) is a graph that illustrates mean-centered first Savitzky-Golay derivative of absorbance derived from transmittance spectra (400-2,450 nm) of 300 raw milk samples, with the most important absorption bands for fat and CP. Aernouts et al. (2011). (Image credits: DOI:https://doi.org/10.3168/jds.2011-4354)

Wavelengths: Based on two studies, the best wavelength suggested here is 930 nm. The study that researched online estimations also recommends 1690 nm, see FIG. 1 above.

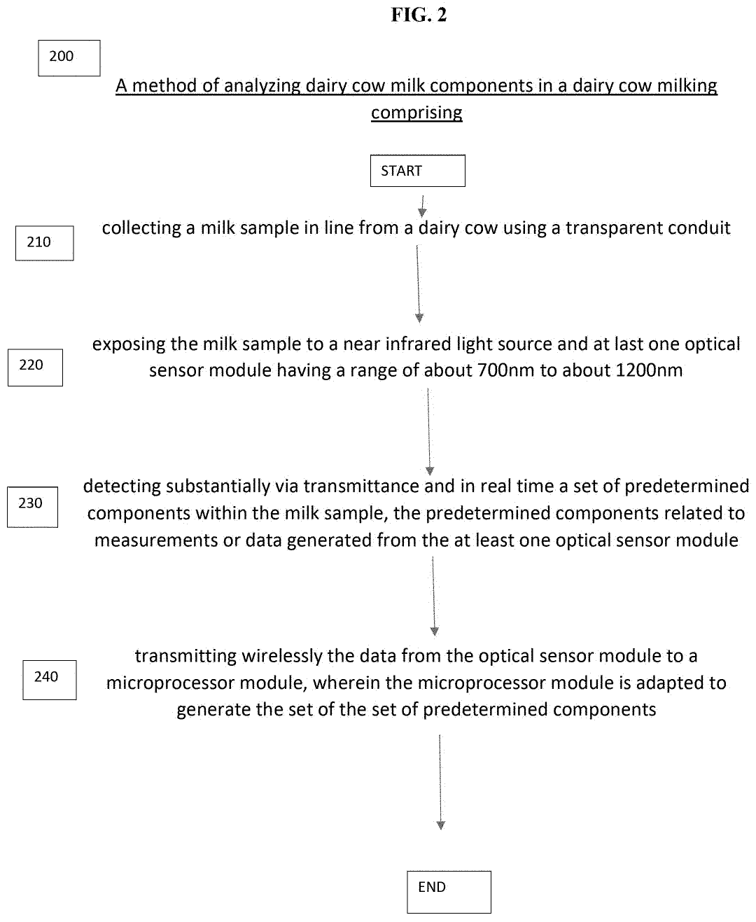

The other study based on at-line estimation recommends 930, 1726, 1760, 2308, 2348, 1160, 1210, 2354 nm; see FIG. 4B (FIG. 2 in the article) is a graph that illustrates milk fat content determination, (Tsenkova et al, 2000). (Image credits: https://doi.org/10.2527/2000.783515x)

Protein Estimation—Mode: Data is collected as transmittance spectra at 2-nm intervals and recorded in the linked computer as absorbance [i.e., log (1/T)]. Wavelengths: Based on the online study the wavelengths recommended are 1650 and 2160 nm, see FIG. 4A (FIG. 1 in the article).

Based on the at-line study the useful wavelengths are 1132, 1460, 1490, 1520, 1990, 2030, and 2070 nm. Also, bands from 1,460 to 1,520 nm, 1,980 to 2,070 nm, and 2,170 to 2,180 nm, respectively, were absorbed by proteins, see FIG. 4C (FIG. 3 in the article) which illustrates the milk total protein content determination. Milk total protein content determination, (Tsenkova et al., 2000). (Image credits: https://doi.org/10.2527/2000.783515x)

Lactose—Mode: Data is collected as transmittance spectra at 2-nm intervals and recorded in the linked computer as absorbance [i.e., log (1/T)]. Wavelengths: Based on the online study the most wavelength recommended is 1490, and also the band between 1480-1500 nm. According to the at-line study, the important wavelengths are 1406 nm, 2011 nm, 1860 nm, and the regions 1438 to 1450 nm, 1460 to 1500 nm, and from 1920 to 2120 nm; see FIG. 4D (see FIG. 4 in the article) illustrating Lactose content determination, (Tsenkova et al, 2000). (Image credits: https://doi.org/10.2527/2000.783515x)

Somatic Cell Count (SCC)—There is only one study that gives details of the wavelengths that is useful for SCC estimation. NIR determination of log SCC was based on relative changes in milk composition affecting milk spectral changes—lactose and proteins. Cows suffering from mastitis produce less lactose, and the type of protein produced changes during illness. Mode: NIR transflectance (T) spectra were collected in a flow cell with a path length of 0.2 mm expressed as absorbance—log(1/T). Wavelength: Real-time analysis is possible by using wavelengths 1412, 1886, 1920,1996, 2020, 2186, 2298, and 2498 nm.

There are a few points that must be kept in mind during inline NIR spectroscopy estimation of milk composition during milking. These are: Milking Dynamics: Expect a shift in spectra from beginning to the end of milking, see FIG. 4E-1 (FIG. 5a in the article). This could be because of a change in the composition of milk, and also due to the mixture of air with milk that scatters light. In the article by Melfsen, FIGS. 4E-E-3 (FIGS. 5a-5c) Near-infrared (NIR) spectra (851 to 1,649 nm) of raw milk from an in-line measurement setup in diffuse reflectance mode. FIG. 4E-2 (FIG. 5a) illustrates typical spectra of milk during 1 cow milking (n=12). FIG. 4E-2 illustrates (FIG. 5b in the article) spectra of all raw milk samples (n=785), while FIG. 4E-3 (FIG. 5c in the article) illustrates normalized NIR reflectance spectra of all raw milk samples (n=785),” (Melfsen et al. 2012). (https://doi.org/10.3168/jds.2012-5388)

Interference by Water: Very low reflectance, absorbance, and transmittance values were recorded for estimation of all parameters, around 960, 970, 1190, 1450, and 1950 nm and above 2400 nm, due to the high absorbance of these wavelengths by water in the milk.

Chemometrics: Water interaction with NIR masks any other interactions. Hence, raw spectra cannot be used to estimate the parameters. The spectral data will have to be pretreated (smoothing and derivative transformation) before using in a chemometrics model, See FIGS. 4E-1 through 4E-3 (FIG. 5b and c in the Melfsen article).

Referring now to FIGS. 1 and 2, FIG. 1 illustrates a schematic of a system 100 of performing an inline real time analysis of a milk sample 14 from a dairy cow 12 within a dairy cow milking system 10. System 100 includes a milk collection vessel or tube or conduit 110 (preferably transparent or translucent) and includes a suction apparatus 120 having an inlet 122 and an outlet 124, the outlet 124 coupled to the milk collection vessel 110 and the inlet 122 designed to be coupled to dairy cow 12. System 100 further includes a near infrared (NIR) spectrometer 130 (which includes a CMOS sensor) designed to provide light and collect light, in a range of about 700 nm to about 1200 nm, to the milk collection vessel 110. Light is collected primarily, but not necessarily limited to, transmittance or absorbance of the near infrared light. System 100 also includes a controller module 140 including a microcontroller 142 and a memory module 144, the controller module 140 designed to receive data from the NIR spectrometer 130, a set of data indicative of data measurements 160 of a set of predetermined milk components.

In another embodiment, system 100 includes a radio frequency (RF) wireless communications module 170 designed to transmit data of at least one of the set of predetermined milk components including fat, protein, lactose, somatic cell contents (SCC), and progesterone to an internal or external network 180 for further processing of data.

Referring now to FIG. 2, there is illustrated a flowchart of a method 200 of collecting a milk sample 14 in line from a dairy cow 12 while on a dairy cow milking system 10. Method 200 includes step 210 of exposing the milk sample 14 to a NIR spectrometer 130 having near infrared light source and an optical sensor. The light source having a range of about 700 nm to about 1200 nm. Step 220 includes detecting in real time a set of predetermined components within the milk sample, such that the predetermined components are detected with a dairy cow milking system 10. In this example embodiment, the set of predetermined milk components includes one or more of protein, fat, progesterone, and somatic cell count. In method 200 the milk components indicate bovine conditions that affect milk production including one or more of mastitis, estrus, dehydration, and starvation.

The following patents are incorporated by reference in their entireties: U.S. Pat. Nos. 8,446,582; and 8,530,830.

While the invention has been described above in terms of specific embodiments, it is to be understood that the invention is not limited to these disclosed embodiments. Upon reading the teachings of this disclosure many modifications and other embodiments of the invention will come to mind of those skilled in the art to which this invention pertains, and which are intended to be and are covered by both this disclosure and the appended claims. It is indeed intended that the scope of the invention should be determined by proper interpretation and construction of the appended claims and their legal equivalents, as understood by those of skill in the art relying upon the disclosure in this specification and the attached drawings.

1. A method of analyzing dairy cow milk components in a dairy cow milking system comprising the steps of:
collecting a milk sample in line from a dairy cow using a transparent conduit;
exposing the milk sample to a near infrared (NIR) light source and at least one optical sensor module having a range of about 700 nm to about 1200 nm;
detecting substantially via transmittance and in real time a set of predetermined components within the milk sample, the predetermined components related to measurements or data generated from the at least one optical sensor module; and
transmitting the data wirelessly from the optical sensor module to a microprocessor module, wherein the microprocessor module is adapted to generate the set of predetermined components.

2. The method as claimed in claim 1, wherein the set of predetermined milk components comprise at least one or more of protein, fat, vitamins, progesterone, and somatic cell count.

3. The method as claimed in claim 1, wherein exposing the milk sample to the NIR light source includes at least one or more wavelengths from a group of wavelengths including: 726, 736, 740, 760, 776, 832, 840, 880, 902, 926, 930, 952, 960, and 1034 nm.

4. The method as claimed in claim 1, wherein exposing the milk sample to the NIR light source includes at least four or more wavelengths from a group of wavelengths including: 726, 736, 740, 760, 776, 832, 840, 880, 902, 926, 930, 952, 960, and 1034 nm.

5. The method as claimed in claim 1, wherein exposing the milk sample to the NIR light source includes one or more wavelengths from a group of wavelengths including: 740 and 840 nm.

6. A method as claimed in claim 1, wherein the milk components indicate bovine conditions that affect milk production, the conditions including one or more of mastitis, estrus, dehydration, and starvation.

7. The method as claimed in claim 1, the method further including conducting chemometrics wherein spectral data is pretreated, such as smoothing and derivative transformation.

8. The method as claimed in claim 1, further including the steps of collecting transmittance spectra at or between about 1-nm to 2-nm intervals, recording at a linked computer as absorbance, and collecting spectral data having a path length up to or between about 9 to 14 mm.

9. A system for analyzing milk components in a dairy cow milking system comprising:
a transparent milk collection vessel or conduit;
a suction apparatus having an inlet and an outlet, the outlet coupled to the milk collection vessel and the inlet adapted to be coupled to a dairy cow;
a near infrared (NIR) spectrometer adapted to provide light to and collect light from, in a range of about 700 nm to about 1200 nm, the milk collection vessel; and
a controller module including a microcontroller and a memory module, the controller module adapted to receive data from the NIR spectrometer indicative of data measurements of a set of predetermined milk components.

10. The system of claim 9 further comprising a radio frequency (RF) wireless communications module operatively coupled to the controller module and adapted to transmit data of at least one of the set of predetermined milk components including fat, protein, lactose, somatic cell contents (SCC), and progesterone.

11. The system of claim 10 wherein the RF module transmits data to a network for further processing in real time.

11. The system of claim 9 wherein the near infrared (NIR) spectrometer is adapted to provide light to and collect light from at least one or more from a group of wavelengths including: 726, 736, 740, 760, 776, 832, 840, 880, 902, 926, 930, 952, 960, and 1034 nm.

12. The system of claim 9 wherein the near infrared (NIR) spectrometer is adapted to provide light to and collect light from at least four or more from a group of wavelengths including: 726, 736, 740, 760, 776, 832, 840, 880, 902, 926, 930, 952, 960, and 1034 nm.

13. The system of claim 9 wherein the near infrared (NIR) spectrometer is adapted to provide light to and collect light from one or more of 740 to 840 nm.

14. The system of claim 9 wherein an online/inline bypass tube of the milk collection vessel is wider than a longest collecting transmittance spectra received.

15. The system of claim 9 having at least one chemometrics model adapted to pretreat raw spectra.

16. A method of analyzing dairy cow milk components in a dairy cow milking system comprising the steps of:
collecting a milk sample in line from a dairy cow using a transparent conduit;
exposing the milk sample to a near infrared (NIR) light source and at least one optical sensor module having a range of about 700 nm to about 1200 nm;
wherein the set of predetermined milk components comprise at least one or more of protein, fat, vitamins, progesterone, and somatic cell count;
detecting substantially via transmittance and in real time a set of predetermined components within the milk sample, the predetermined components related to measurements or data generated from the at least one optical sensor module;
collecting transmittance spectra at or between about 1-nm to 2-nm intervals, recording in a linked computer as absorbance;
collecting spectral data having a path length up to at or between about 9 to 14 mm;
collecting transmittance spectra received through an online/inline bypass tube of the milk collection vessel wherein the collecting transmittance spectra is wider than the bypass tube; and
transmitting the data from the optical sensor module to a microprocessor module, wherein the microprocessor module is adapted to generate the set of predetermined components.

17. The method as claimed in claim 16, wherein exposing the milk sample to the NIR light source includes at least one or more wavelengths from a group of wavelengths including: 726, 736, 740, 760, 776, 832, 840, 880, 902, 926, 930, 952, 960, and 1034 nm.

18. The method as claimed in claim 16, wherein exposing the milk sample to the NIR light source includes one or more wavelengths from a group of wavelengths including: 740 and 840 nm.

19. A method as claimed in claim 16, wherein the milk components indicate bovine conditions that affect milk production, the conditions including one or more of mastitis, estrus, dehydration, and starvation.

20. The method as claimed in claim 16, the method further including conducting chemometrics wherein spectral data is pretreated, such as smoothing and derivative transformation.
